(19) United States
(12) Patent Application Publication
(10) Pub. No.: US 2008/0159995 A1
(43) Pub. Date: Jul. 3, 2008

(54) TREATMENT OF FIBROSIS

(76) Inventors: Monica Silverstone Spiteri, Stoke-on-Trent (GB); Keira Louise Watts, Newcastle under Lyme (GB)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/866,511

(22) Filed: Oct. 3, 2007

Related U.S. Application Data
(60) Provisional application No. 60/867,446, filed on Nov. 28, 2006.

(30) Foreign Application Priority Data
Oct. 3, 2006 (GB) 0619500.2

(51) Int. Cl. A61K 35/56; A61K 31/351; A61K 31/404; A61K 31/435; A61P 19/04; A61K 31/505; A61K 31/47; A61K 31/40

(52) U.S. Cl. 424/93.7; 514/460; 514/419; 514/277; 514/275; 514/311; 514/423

(57) ABSTRACT

The present invention relates to methods and compositions for treating fibrosis. In one aspect the invention provides the use of an agent capable of increasing the number of stem cells and/or progenitor cells available to and/or engraftment at a site of fibrosis in the manufacture of a medicament for use in conjunction with an anti-fibrotic agent for the treatment of fibrosis. The anti-fibrotic agent can be supplied to the subject before, at the same time, or after the agent capable of increasing the number of stem cells and/or progenitor cells available to and/or engraftment at a site of fibrosis. The fibrosis may be idiopathic pulmonary fibrosis. The anti-fibrotic agent can be a modulator of RhoA, RhoA GTPases, TGF-β1 or CTGF, or any other member of the RhoA signalling pathway; or can modulate the effect of suppressor of cytokine signalling 1 (SOCS 1), suppressor of cytokine signalling 3 (SOCS 3) or TLR9; or can be a statin compound or derivative thereof.

### RELATED APPLICATION DATA

This application claims the benefit of UK Patent Application GB 0619500.2 filed Oct. 3, 2006 and U.S. Provisional Application Ser. No. 60/867,446 filed Nov. 28, 2006, the disclosures of which are incorporated by reference.

### BACKGROUND

The present invention relates to methods and compositions for treating fibrosis.

Fibrosis is a condition characterised by the formation or development of excess fibrous connective tissue, excess extracellular matrix (ECM), excess scarring or excess collagen deposition in an organ or tissue as a reparative or reactive process. Fibrosis related diseases include: idiopathic pulmonary fibrosis; skin fibrosis, such as scleroderma, post-traumatic and operative cutaneous scarring; eye fibrosis, such as sclerosis of the eyes, conjunctival and corneal scarring, pterygium; cystic fibrosis of the pancreas and lungs; endomyocardial fibrosis; idiopathic myocardiopathy; cirrhosis; mediastinal fibrosis; progressive massive fibrosis; proliferative fibrosis; neoplastic fibrosis. Tuberculosis may cause fibrosis of the lungs.

Idiopathic pulmonary fibrosis describes a group of diseases whereby scarring occurs in the interstitium (or parenchymal) tissue of the lung; this tissue supports the air-sacs or alveoli. During idiopathic pulmonary fibrosis, these air sacs become replaced by fibrotic tissue, causing the tissue to become restructured. This results in a disruption to the alveolar-capillary interface, a loss of tissue function, reducing the ability of the lung to transfer oxygen into the bloodstream. This relentless disease causes progressive structural remodelling of the lungs and is characterised clinically, for example, by increasing shortness of breath, chronic cough, progressive reduction in exercise tolerance and general fatigue. The disease can progress over a period of years, or progress very rapidly, resulting in patient debility, respiratory failure and eventually death.

Development of fibrosis within the lungs can occur within the bronchial walls of chronic inflammatory airway diseases such as asthma, COPD (chronic obstructive pulmonary disease), emphysema, and chronic smokers' airways. Furthermore, the presence and persistence of fibrosis both in the lung parenchyma (interstitium) and within the bronchial airway walls causes in situ structural remodelling leading to the lung losing its delicate anatomical function and respiratory capability.

Idiopathic pulmonary fibrosis has been linked to a number of different causes, including autoimmune disorders, genetic predisposition, and prolonged exposure to occupational, environmental and/or inhaled contaminants, e.g. dust; viruses, gastro-oesopageal reflux. Idiopathic pulmonary fibrosis affects more than 5 million people worldwide, with an estimated 50,000 new cases this year, which is anticipated to increase −40,000 deaths from this disease was reported in 2005. The prognosis of this disease is poor-average expected life span is 2.9 years from diagnosis. However clinical outcome is dependant upon age, lifestyle, mode of initial presentation and histological staging. The average age of onset is between 40-60 years, however there are some reported cases in children as young as 3.

Marked disruption in airway epithelial cell (AEC) integrity is the hallmark of idiopathic pulmonary fibrosis. This reflects abnormal wound repair, possibly due to inappropriate epithelial-mesenchymal signalling and aberrant lung stem cell differentiation. Sequential microinjury/ies provoke marked disruption in AEC integrity with diverse hyperplastic/metaplastic phenotypes in situ. Adjacent are distinct ‘fibroblastic foci’ of actively proliferating and secreting fibroblasts/myofibroblasts, with aberrant collagen synthesis and exaggerated ECM deposition. Pulmonary stem cells, whether resident niches or recruited to the lung, have a capability for both epithelial and mesenchymal lineage potential.

It remains unclear why endogenous progenitor cells fail to regenerate alveolar epithelial tissue in idiopathic pulmonary fibrosis, and whether this is due to inappropriate terminal cell differentiation or apoptosis. In respect of the former, possibilities include: (i) aberrant epithelial-mesenchymal transdifferentiation to restore epithelial cell—fibroblast balance; (ii) fibroblast/myofibroblast development from a reservoir of resident tissue-specific precursors; (iii) fibroblast phenotypes arising from progenitor cells. All are plausible mechanisms and may co-exist; their presence regulated by the particular pathogenic stage of the local milieu.

To date, there are no efficacious therapies available that can either halt or reverse the pro-fibrogenic processes in the lungs. Conventional therapy utilises single or combined use of immunosuppressive and/or anti-inflammatory agents such as corticosteroids, azathioprine, cyclosprin, cyclophosphamide, methotrexate, hydroxychloroquine, with negligible to varied limited clinical response; none appear to have an effect on stopping and/or slowing fibrosis development, promoting lung repair and restoration to normal and/or near-normal function. Thus patients invariably deteriorate with irreversible respiratory failure (Walter et al (2006) Proc Am Thorac Soc 3, 330-338). While donated healthy lung transplants have been used to replace fibrosed lungs, the clinical need for such transplants far outweighs available supply.

Thus there is a need for new and efficacious treatments for fibrosis and particularly idiopathic pulmonary fibrosis.

### DETAILED DESCRIPTION

Although the characteristic features of this invention will be particularly pointed out in the claims, the invention itself, and the manner in which it may be made and used, may be better understood by referring to the following description taken in connection with the accompanying figures forming a part thereof.

A first aspect of the invention provides the use of an agent capable of increasing the number of stem cells and/or progenitor cells available to and/or engraftment at a site of fibrosis in the manufacture of a medicament for use in conjunction with an anti-fibrotic agent for the treatment of fibrosis.

A further aspect of the invention provides a method of treating fibrosis comprising administering an agent capable of increasing the number of stem cells and/or progenitor cells available to and/or engraftment at a site of fibrosis in conjunction with an anti-fibrotic agent, to a subject in need of said administration.

The “agent capable of increasing the number of stem cells and/or progenitor cells available to and/or engraftment at a site of fibrosis” can be any molecule or cell that can be used for this purpose. In one embodiment of the invention, the agent is a therapeutically effective quantity of stem cells and/or progenitor cells. Therefore the invention may comprise supplying a subject with a quantity of stem/progenitor cells. However, the invention also encompasses where the “agent capable of increasing the number of stem cells and/or progenitor cells available to and/or engraftment at a site of fibrosis” increases the amount of endogenous stem cells and/or progenitor cells.

By “increasing the number of stem cells and/or progenitor cells”, we include where the agent increases the actual number of stem or progenitor cells recruited to a site of fibrosis. However, we also encompass where the agent does not increase the number of stem or progenitor cells, but rather existing stem or progenitor cells are more able to engraft at the site of fibrosis and so to therapeutically contribute to the treatment of fibrosis. For example, the agent promotes stem or progenitor cells to produce functional cells and/or tissues, thereby restoring normal, or near normal, healthy activity or function. Therefore, by “engraftment at” we include where the agent increases the number of stem or progenitor cells that engraft at the site of fibrosis.

By “a site of fibrosis” we mean the site of fibrosis in the subject to be treated. Therefore, the actual location in the body will be dependent on the particular fibrotic disorder the subject has developed. For example, where the subject to be treated has idiopathic pulmonary fibrosis, then the site of fibrosis will be the lung parenchyma. In other instances such as chronic inflammatory airways disease, the site of fibrosis may be the bronchial airways/airspaces.

By “in conjunction with” we include where the anti-fibrotic agent is administered to a subject before, at the same time, or after as the agent capable of increasing the number of stem cells and/or progenitor cells available to and/or engraftment at a site of fibrosis is administered to the subject. Also, where the medicament or method includes a therapeutically effective quantity of stem cells and/or progenitor cells, then “in conjunction with” can include where the stem cells and/or progenitor cells are pre-incubated with the anti-fibrotic agent prior to being administered to the subject.

In an embodiment of the invention, the agent capable of increasing the number of stem cells and/or progenitor cells available to and/or engraftment at a site of fibrosis is supplied to a subject at the same time as the anti-fibrotic agent. Preferably, in such an embodiment the medicament comprises a combination of both an agent capable of increasing the number of stem cells and/or progenitor cells available to and/or engraftment at a site of fibrosis, and an anti-fibrotic agent.

In an alternative embodiment of the invention the agent capable of increasing the number of stem cells and/or progenitor cells available to and/or engraftment at a site of fibrosis is administered to a subject who has previously been administered an anti-fibrotic agent. As will be appreciated by the skilled person, the length of the delay in administration of the two components of the treatment is dependent on the nature of the anti-fibrotic agent used. For example, where the anti-fibrotic agent is only effective for a short period of time in the body of the subject, then the agent capable of increasing the number of stem cells and/or progenitor cells available to and/or engraftment at a site of fibrosis would have to be supplied to the subject shortly after, and while the anti-fibrotic agent was still effective.

By “anti-fibrotic agents” we include agents that act to reduce fibrosis in a subject. Examples of anti-fibrotic agents are discussed further below and include modulators of RhoA, RhoA GTPases, TGF-β1 and CTGF signalling molecules, or other members of the RhoA signalling pathway. Such agents also include agents that modulate the effect of suppressor of cytokine signalling 1 (SOCS 1), suppressor of cytokine signalling 3 (SOCS 3), or of TLR9, a SOCS 3 receptor. A preferred embodiment of the invention is where the anti-fibrotic agent is a statin compound or derivative thereof.

By “subject” we include any animal that is susceptible to developing fibrosis, preferably a vertebrate, more preferably a mammal such as a domesticated farmyard animal or a human being. Most preferably the subject is a human being.

By “fibrosis” we include any condition characterised by the formation or development of excess fibrous connective tissue, excess extracellular matrix, excess scarring or excess collagen deposition in an organ or tissue as a reparative or reactive process. Fibrosis related diseases include: idiopathic pulmonary fibrosis; skin fibrosis, such as scleroderma, post-traumatic and operative cutaneous scarring; eye fibrosis, such as sclerosis of the eyes, conjunctival and corneal scarring, pterygium; cystic fibrosis of the pancreas and lungs; endomyocardial fibrosis; idiopathic myocardiopathy; cirrhosis; mediastinal fibrosis; progressive massive fibrosis; proliferative fibrosis; neoplastic fibrosis. Tuberculosis may cause fibrosis of the lungs. Therefore the present invention can be used to treat fibrosis in a wide range of organs and tissues, including the lung, eye, skin, kidney, liver, pancreas and joints.

Preferably the fibrosis is idiopathic pulmonary fibrosis. Idiopathic pulmonary fibrosis is a group of diseases whereby scarring occurs in the interstitium (or parenchyma) tissue of the lung. Development of fibrosis in the lungs can also occur within the bronchial walls of chronic inflammatory airway diseases such as asthma, COPD (chronic obstructive pulmonary disease), emphysema, and smokers' lung.

In an alternative embodiment of the invention the fibrosis is eye or skin fibrosis. Examples of skin fibrotic disorders include scleroderma, post-traumatic and operative cutaneous scarring. Examples of fibrotic disorders of the eye include sclerosis of the eyes; conjunctival and corneal scarring; pterygium.

The present invention relates to the novel and surprising finding that anti-fibrotic agents can act to promote stem or progenitor cells to differentiate in situ into therapeutic cell types. While not wishing to be limited to any particular theory, the inventors believe that the anti-fibrotic agents may act to limit fibrosis at a locality and thus establish a local milieu in which stem or progenitor cells may successfully engraft and contribute to the healing response. That a local non-fibrotic milieu may assist to allow stem or progenitor cells to differentiate in situ into therapeutic cell types had not been suggested prior to the invention. Moreover, it had not been previously suggested to combine anti-fibrotic agents with stem cells and/or progenitor cells to improve the effectiveness of stem or progenitor cell therapy. This surprising effect leads to an effective treatment for fibrosis.

Stem cells are cells that have the potential to differentiate into a number of cell types in the body. Theoretically, stem cells may divide without limit to replenish other cells for as long as the organism is alive. Upon differentiation, the daughter cell has the potential to remain a stem cell or become another cell type, for example lung cell and display its characteristics, thus holding promise for many diseases by replacing damaged tissues. These phenomena may be induced under specific physiological and experimental conditions.

In general, stem cell therapy represents a therapeutic method by which degenerative and/progressive diseases (such as those caused by premature death or malfunction of cell types that the body is unable to replace) may be treated. It is hoped that addition of stem cells may help nucleate and promote the development of functional cells and/or tissues to replace those lost, thereby restoring normal healthy activity/function.

For the purposes of the present invention, “stem cells” are taken to comprise nullipotent, totipotent or pluripotent cells, and progenitor cells (or precursor cells) to comprise multipotent cells. For the avoidance of doubt, the medicament and methods of the invention can comprise a therapeutically effective quantity of either stem or progenitor cells, or both stem and progenitor cells.

Totipotent cells are those cells capable of giving rise to all extraembryonic, embryonic and adult cells of the embryo. Accordingly it can be seen that totipotent cells may ultimately give rise to any type of differentiated cell found in an embryo or adult. By comparison, pluripotent cells are cells capable of giving rise to some extraembryonic and all embryonic and adult cells. Thus it can be seen that pluripotent cells are able to give rise to a more limited range of cell types than are totipotent cells. Nullipotent cells are those that will not undergo differentiation without the action of an exogenous cue to differentiation. Multipotent cells are cells able to give rise to diverse cell types in response to appropriate environmental cues (such as action of soluble growth factors or the substrate on which the cell, or its progeny, is located), but are more restricted in their potential lineage formation than are pluripotent, nullipotent or totipotent cells.

Preferred culture conditions for use in accordance with the present invention may be determined with reference to the type of biological cell to be cultured. Consideration should be given both to the nature of the cell (e.g. stem or progenitor cell), to the source of the cell, and also to the manner in which the cell is to be utilised. Suitable culture conditions are well known to those skilled in the art.

A suitable source of stem cells that may be used in accordance with the present invention are cells derived from the inner cell mass/epiblast of pre-implantation embryos. Such embryonic stem (ES) cells are readily obtainable and are capable of giving rise to all possible embryonic and adult cell lineages. In particular, the undifferentiated human ESC (H1 line from WiCell Research Institute, Inc, Madison, Wis.: www.wicell.org) could be used in the invention; this cell line is commercially available. A further source of stem cells that can be used in the present invention are umbilical cord-derived cells.

Undifferentiated embryonic stem cells (ESC) from established ethical cell lines like H1, or umbilical cord-derived cells, can be supplied to subjects in an undifferentiated form. Alternatively, the ESCs and mesenchymal stem cells (MSCs) can be differentiated prior to patient engraftment but not necessarily purified, or differentiated and purified prior to engraftment. With reference to mesenchymal stem cells, it would be possible to obtain MSCs from the subject to be treated. Such MSCs could be encouraged to differentiate into alveolar epithelial cells prior to administration.

Progenitor cells can also be used in the present invention. Progenitor cells arise from division of stem cells but are limited in the number of cell division cycles they can go through. They divide rapidly to produce a pool of cells that then differentiate and integrate into the tissue. An example of a progenitor cell type that can be used in the invention is mesenchymal stem cells, or marrow stromal cells (MSC). MSCs are multipotent stem cells that can differentiate into a variety of cell types. The type of progenitor cell to be used may be dependent on the fibrotic disorder to be treated.

It is usual practice to select progenitor cells from a population of cells on the basis of specific cell markers, as would be appreciated by the skilled person. Therefore, for example a progenitor cell suitable for use in treating idiopathic pulmonary fibrosis would preferably be encouraged to differentiate down the alveolar epithelial cell lineage. Such differentiation is marked by the presence of lineage markers on the cell surface, for example: expression of laminin 5 and surfactant proteins; in addition TTF-1 (distal lung epithelium), CC10 (Clara cells) and Aquaporin 5+Tlalpha (type I cells) will also be assessed as a broad spectrum of lung epithelial lineages. Such cell surface markers are distinct from pro-fibroblast/myofibroblast lineage expression markers, e.g. α-SMA, pro-collagen I and III.

It will be appreciated that the precise nature of the biological cell selected for use in accordance with the invention may be determined on the basis of the therapeutic use to which the cell is to be put. For example, in therapy of the lung it may be preferred to utilise a biological cell derived from the lung; where it is wished to effect therapy of the epidermis it will be preferred to use an epidermal cell; where it is wished to effect therapy of the eye it will be preferred to use an ocular-related cell relevant to site of repair, for example cornea, conjunctiva. Thus the cells to be cultured will generally be selected based on the therapy to be effected. Suitable protocols for the harvesting of biological cells for use in accordance with the therapeutic applications of the invention will vary according to the source of the cells to be used. Cell harvest protocols are well known, and preferred protocols may be readily determined by those skilled in the art.

Where the medicament or method of the invention is to treat idiopathic pulmonary fibrosis, it is preferred that the stem or progenitor cell/s to be used has been encouraged to differentiate into an airway epithelial progenitor cell. Preferably the stem or progenitor cell used in the invention will differentiate into an alveolar epithelial cell. The differentiated stem or progenitor cells may or may not be purified from the undifferentiated stem or progenitor cells prior to supply to a subject.

As would be appreciated by the skilled person, the methods used to diagnose fibrotic diseases are dependent on the particular type of fibrosis to be assessed. For example, where the fibrosis is idiopathic pulmonary fibrosis then diagnosis may progress by a high resolution CT scan of the chest, which can be supported by a lung biopsy. Using this diagnostic assessment a subject can be selected for treatment with a medicament or method of the invention. Further information regarding diagnosis of idiopathic pulmonary fibrosis may be found in Demedts and Costabel (2002) Eur Respir J 19, 794-796. Methods of diagnosing skin and eye-related fibrotic disorders are similarly well known.

In the methods of therapy according to the present invention, and in the use of medicaments according to the invention, a therapeutically effective quantity of stem or progenitor cells can be administered to a subject requiring therapy. A “therapeutically effective quantity” in the context of the present invention is considered to be any quantity of suitable cells which, when administered to a subject suffering from a fibrotic disease against which the cells are effective, causes reduction, remission, or regression of the fibrosis.

As discussed above, the “agent capable of increasing the number of stem cells and/or progenitor cells available to and/or engraftment at a site of fibrosis” can be an agent that acts to increase the amount of endogenous stem cells and/or progenitor cells.

By “increase the amount of endogenous stem cells and/or progenitor cells” we include where the agent increases the number of endogenous stem or progenitor cells, or increases the engraftment of such cells, by 2×, 3×, 5×, 10×, 20×, 50× or 100×.

Alternatively, the “agent capable of increasing the number of stem cells and/or progenitor cells available to and/or engraftment at a site of fibrosis” can comprise both a therapeutically effective quantity of stem cells and/or progenitor cells and an agent that increases the amount of endogenous stem cells and/or progenitor cells.

As discussed above, by “anti-fibrotic agents” we include agents that act to reduce fibrosis in a subject. Examples of anti-fibrotic agents are discussed further below and include modulators of RhoA, RhoA GTPases, TGF-β1 and CTGF signalling molecules, or other members of the RhoA signalling pathway such as Endothelial-1 (ET-1). Such agents also include agents that modulate the effect of suppressor of cytokine signalling 1 (SOCS 1), suppressor of cytokine signalling 3 (SOCS 3), or of TLR9, a SOCS 3 receptor.

The inventors have recently shown that RhoA plays a central role in the pathogenesis of fibrotic lung disease. Similarly, Rho GTPases have been shown to play a pivotal role in the activation of transforming growth factor-β1 (TGF-β1) and downstream signalling molecules, including connective tissue growth factor (CTGF), which are central fibrogenic factors responsible for collagen production, ECM deposition and mobilisation of other essential pro-fibrotic cellular/soluble components. Therefore agents that modulate the effect of RhoA, RhoA GTPases, TGF-β1 and CTGF signalling molecules are considered to be anti-fibrotic agents.

Specific targeting of RhoA, or its manipulation to inhibit its activation, can provide novel anti-fibrotic treatments. From this work the inventors have demonstrated that statins can be used as anti-fibrotic agents, most likely by acting as modulators of RhoA or RhoA GTPases activity.

Accordingly, in one embodiment of the medicament and methods of the invention the anti-fibrotic agent is a statin compound or derivative thereof.

Statins are a widely prescribed group of drugs, predominately used to lower blood cholesterol levels. Statins are known to interfere with the synthesis of cholesterol, by blocking the enzyme-3-hydroxy-3-methylglutaryl coenzyme A reductase.

Statins were the second-best selling class of drugs worldwide in 2000, with nearly $16 billion in global sales. In the U.S., statin sales increased from $3.6 billion in 1997 to $9 billion in 2000. About 48 million prescriptions were written for atorvastatin alone in 2000, making it the most frequently dispensed drug in the U.S. that year. Statins include lovastatin, pravastatin, fluvastatin, cerivastatin, atorvastatin, simvastatin, pitavastatin and rosuvastatin.

Lovastatin: systematic (IWPAC) name of [8-[2-(4-hydroxy-6-oxo-oxan-2-yl)ethyl]-3,7-dimethyl-1,2,3,7,8,8a-hexahydronaphthalen-1-yl]2-methylbutanoate. It has the molecular formula of C₂₄H₃₆O₅ and a molecular weight of 404.54 g/mol. Lovastatin is a white, nonhygroscopic crystalline powder that is insoluble in water and sparingly soluble in ethanol, methanol, and acetonitrile.

Pravastatin: systematic (IUPAC) name of 3,5-dihydroxy-7-[6-hydroxy-2-methyl-8-(2-methylbutanoyloxy)-1,2,6,7,8,8a-hexahydronaphthalen-1-yl]-heptanoic acid. It has the molecular formula of C₂₃H₃₆O₇ and a molecular weight of 424.528 g/mol.

Fluvastatin: systematic (IUPAC) name of 7-[3-(4-fluorophenyl)-1-(1-methylethyl)-1H-indol-2-yl]-3,5-dihydroxy-hept-6-enoic acid. It has the molecular formula of C₂₄H₂₆FNO₄ and a molecular weight of 411.466 g/mol.

Cerivastatin: systematic (IUPAC) name of (E,3R,5S)-7-[4-(4-fluorophenyl)-5-(methoxymethyl)-2,6-dipropan-2-yl-pyridin-3-yl]-3,5-dihydroxy-hept-6-enoic acid. It has the molecular formula of C₂₆H₃₄FNO₅ and a molecular weight of 459.55 g/mol.

Atorvastatin: systematic (IUPAC) name of [R—(R*, R*)]-2-(4-fluorophenyl)-beta, delta-dihydroxy-5-(1-methylethyl)-3-phenyl-4-[(phenylamino)carbonyl]-1H-pyrrole-1-heptanoic acid. It has the molecular formula of C₃₃H₃₄FN₂O₅ and a molecular weight of 558.64 g/mol.

Simvastatin: systematic (IUPAC) name of [(1S,3R,7R,8S,8aR)-8-[2-[(2R,4R)-4-hydroxy-6-oxo-oxan-2-yl]ethyl]-3,7-dimethyl-1,2,3,7,8,8a-hexahydronaphthalen-1 yl]2,2-dimethylbutanoate. It has the molecular formula of C₂₅H₃₈O₅ and a molecular weight of 418.566 g/mol.

Pitavastatin: systematic (IUPAC) name of (E)-7-[2-cyclopropyl-4-(4-fluorophenyl)

quinolin-3-yl]-3,5-dihydroxy-hept-6-enoic acid. It has the molecular formula of C₂₅H₂₄FNO₄ and a molecular weight of 421.461 g/mol.

Rosuvastatin: systematic (IUPAC) name of 7-[4-(4-fluorophenyl)-6-(1-methylethyl)-2-(methyl-methylsulfonyl-amino)-pyrimidin-5-yl]-3,5-dihydroxy-hept-6-enoic acid. It has the molecular formula of C₂₂H₂₈N₃FO₆S and a molecular weight of 481.539 g/mol.

The amount of the statin compound or derivative thereof that can be used in the medicament or method of the invention can be determined using the assay for anti-fibrotic activity outlined herein. Also, the therapeutically effective quantity of statins are well known and can be obtained from the appropriate supplier or, for example, the US Food and Drink Association (US FDA) website: www.fda.gov.

Preferably the statin is simvastatin.

By “statin or a derivative thereof” we include structurally related compounds able to modulate RhoA or RhoA GTPases activity. We also include prodrug forms of statins. Moreover, the nanoparticulated forms of statins discussed below are also considered to be derivatives of statin compounds. Example 1 below provides an assay by which the effect of a compound on fibrosis can be assessed. Therefore, the assay can be used to determine whether a structurally related compound to a statin can still function to modulate RhoA or RhoA GTPases activity. The term “prodrug” as used in this application refers to a precursor or derivative form of a statin that is capable of being enzymatically activated or converted into the more active parent form.

As discussed above, agents that modulate the effect of RhoA, RhoA GTPases, TGF-β1 and CTGF signalling molecules, or other members of the RhoA signalling pathway, can be used in the present invention as anti-fibrotic agents. By “modulator” we preferably mean that the agent reduces the amount or function of the molecule. However, the agent could also override the effect of that molecule in the signalling pathway.

By “reduces the amount or function” we include where the agent reduces the amount or function of the signalling molecule by 2×, 3×, 5×, 10×, 20×, 50×, or 100× the natural amount or function.

Such agents may include, for example, antisense molecules, ribozymes, and antagonists such as neutralising antibodies to the specific signalling molecules.

The RhoA, RhoA GTPases, TGF-β1 and CTGF signalling molecules are well known in the art. The skilled person can readily identify nucleic acid and polypeptide sequences corresponding to each of these signalling molecules. For example, RhoA sequence is provided in UniProt accession P06749/Q9UEJ4. TGF-β1 can be found at UniProt accession P01137/Q9UCG4. CTGF can be found at UniProt accession P29279/Q96QX2.

Searches of the UniProt database can be conducted using the URL: http://www.ebi.uniprot.org/uniprot-srv/index.do

Antisense oligonucleotides are single-stranded nucleic acids, which can specifically bind to a complementary nucleic acid sequence. By binding to the appropriate target sequence, an RNA-RNA, a DNA-DNA, or RNA-DNA duplex is formed. These nucleic acids are often termed “antisense” because they are complementary to the sense or coding strand of the gene. Recently, formation of a triple helix has proven possible where the oligonucleotide is bound to a DNA duplex. It was found that oligonucleotides could recognise sequences in the major groove of the DNA double helix. A triple helix was formed thereby. This suggests that it is possible to synthesise sequence-specific molecules which specifically bind double-stranded DNA via appropriate formation of major groove hydrogen bonds.

By binding to the target nucleic acid, the above oligonucleotides can inhibit the function of the target nucleic acid. This could, for example, be a result of blocking the transcription, processing, poly(A)addition, replication, translation, or promoting inhibitory mechanisms of the cells, such as promoting RNA degradations.

Antisense oligonucleotides are prepared in the laboratory using standard laboratory protocols, as would be appreciated by the skilled person. The antisense molecule can then be supplied to a subject by, for example, airway delivery. Antisense oligonucleotides were first discovered to inhibit viral replication or expression in cell culture for Rous sarcoma virus, vesicular stomatitis virus, herpes simplex virus type 1, simian virus and influenza virus. Since then, inhibition of mRNA translation by antisense oligonucleotides has been studied extensively in cell-free systems including rabbit reticulocyte lysates and wheat germ extracts. Inhibition of viral function by antisense oligonucleotides has been demonstrated in vitro using oligonucleotides which were complementary to the AIDS HIV retrovirus RNA.

Typically, antisense oligonucleotides are 15 to 35 bases in length. For example, 20-mer oligonucleotides have been shown to inhibit the expression of the epidermal growth factor receptor mRNA, and 25-mer oligonucleotides have been shown to decrease the expression of adrenocorticotropic hormone by greater than 90%. However, it is appreciated that it may be desirable to use oligonucleotides with lengths outside this range, for example 10, 11, 12, 13, or 14 bases, or 36, 37, 38, 39 or 40 bases. The design of antisense molecules to nucleic acid corresponding to RhoA, RhoA GTPases, TGF-β1 and CTGF signalling molecules, or other members of the RhoA signalling pathway, is routine and can readily be performed by the skilled person.

By “antisense” we also include all methods of RNA interference, which are regarded for the purposes of this invention as a type of antisense technology.

A further method of modulating RhoA, RhoA GTPases, TGF-β1 and CTGF signalling molecules, or other members of the RhoA signalling pathway, is to use a ribozyme capable of cleaving RNA or DNA encoding these proteins. A gene expressing said ribozyme, or ribozyme protein, may be administered in substantially the same and using substantially the same vehicles as for antisense molecules. It will be appreciated that it may be desirable that the antisense molecule or ribozyme may be expressed from a cell-specific promoter element, or a regulatable promoter.

Such genetic constructs of the invention can be prepared using methods well known in the art.

A further method of modulating RhoA, RhoA GTPases, TGF-β1 and CTGF signalling molecules, or other members of the RhoA signalling pathway, is to use one or more agents that act as antagonists to these polypeptides.

The term “antagonist” is well known to those skilled in the art. By “antagonist” we include in this definition any agent that acts to alter the level and/or functional ability of RhoA, RhoA GTPases, TGF-β1 and CTGF signalling molecules, or other members of the RhoA signalling pathway. An example of an antagonist would include a chemical ligand that binds to and affects polypeptide function, and in broader terms this could also include an antibody, or antibody fragment, that binds to one of the said polypeptides such that the polypeptide cannot affect its normal function. The antagonist may also alter the sub-cellular localisation of the polypeptide; in this way, the amount of functional polypeptide is reduced.

By “antibody” we include intact monoclonal and polyclonal antibody molecules as well as antibody fragments (such as, for example, Fab and F(ab′)2 fragments). Fab and F(ab′)2 fragments lack the Fc fragment of intact antibody, clear more rapidly from the circulation, and may have less non-specific tissue binding of an intact antibody.

A further method of modulating RhoA, RhoA GTPases, TGF-β1 and CTGF signalling molecules, or other members of the RhoA signalling pathway, is to use a dominant inactive form of the molecule. For example a polypeptide may be modified so as to generate a dominant inactive form of a RhoA or RhoA GTPase, but cannot function in the Rho signalling pathway. Alternatively the dominant inactive form may not be correctly trafficked within the cell.

Methods of modifying polypeptide sequence are commonplace in the art and can be readily utilised by the skilled person. The effect of a modification in polypeptide sequence on the function of that polypeptide can be assessed using the assay set out in Example 1 below.

Further “anti-fibrotic agents” include agents that modulate the effect of suppressor of cytokine signalling 1 (SOCS 1), suppressor of cytokine signalling 3 (SOCS 3), or of TLR9, a SOCS 3 receptor. By “modulator” we preferably mean that the agent increases the amount or function of that molecule, or overrides cell-cell or cell-mediator interaction so as to make an effect of an increase in the amount or function of that molecule.

SOCS 3 expression has been shown to be absent and/or downregulated in fibrotic conditions in organs including the liver. The inventors have also shown that SOCS 3 expression is similarly deregulated in lung fibrosis. Therefore in one embodiment of the medicament and methods of the invention the anti-fibrotic agent is an activator of SOCS 3 expression or activity.

SOCS 1 and SOCS 3 are members of the STAT-induced STAT inhibitor (SSI), also known as suppressor of cytokine signalling (SOCS) family. SSI family members are cytokine-inducible negative regulators of cytokine signalling.

The protein sequence of human SOCS 1 and SOCS 3 is provided below. Further information regarding the protein and nucleic acid sequence can be found at the UniProt accession.

SOCS 1 protein sequence (UniProt accession o15524;
o15097; q9nsa7):
MVAHNQVAAD NAVSTAAEPR RRPEPSSSSS SSPAAPARPR
PCPAVPAPAP GDTHFRTFRS HADYRRITRA SALLDACGFY
WGPLSVHGAH ERLRAEPVGT FLVRDSRQRN CFFALSVKMA
SGPTSIRVHF QAGRFHLDGS RESFDCLFEL LEHYVAAPRR
MLGAPLRQRR VRPLQELCRQ RIVATVGREN LARIPLNPVL
RDYLSSFPFQ I
SOCS 3 protein sequence (UniProt accession o14543;
o14509):
MVTHSKFPAA GMSRPLDTSL RLKTFSSKSE YQLVVNAVRK
LQESGFYWSA VTGGEANLLL SAEPAGTFLI RDSSDQRHFF
TLSVKTQSGT KNLRIQCEGG SFSLQSDPRS TQPVPRFDCV
LKLVHHYMPP PGAPSFPSPP TEPSSEVPEQ PSAQPLPGSP
PRRAYYIYSG GEKIPLVLSR PLSSNVATLQ HLCRKTVNGH
LDSYEKVTQL PSPIREFLDQ YDAPL

In one embodiment the anti-fibrotic agent can be SOCS 1 or SOCS 3 polypeptide, or a functional fragment or variant of thereof, or a fusion thereof. Methods of synthesising SOCS 1 and SOCS 3 polypeptide would be routine to the person skilled in the art.

Anti-fibrotic agents also include agents that can increase the amount or function of TLR9, a receptor for the SOCS 3 signalling molecule.

TLR9 is a member of the Toll-like receptors for ligands. The protein sequence of human TLR9 is provided below. Further information regarding the protein and nucleic acid sequence can be found at the UniProt accession.

TLR 9 protein sequence (UniProt accession q9nr96;
q6uvz2; q9hd68; q9hd69; q9hd70; q9nyc2; q9nyc3):
MSFCRSALHP LSLLVQAIML AMTLALGTLP AFLPCELQPH
GLVNCNWLFL KSVPHFSMAA PRGNVTSLSL SSNRIHHLHD
SDFAHLPSLR HLNLKWNCPP VGLSPMHFPC HMTIEPSTFL
AVPTLEELNL SYNNIMTVPA LPKSLISLSL SHTNILMLDS
ASLAGLHALR FLFMDGNCYY KNPCRQALEV APGALLGLGN
LTHLSLKYNN LTVVPRNLPS SLEYLLLSYN RIVKLAPEDL
ANLTALRVLD VGGNCRRCDH APNPCMECPR HFPQLHPDTF
SHLSRLEGLV LKDSSLSWLN ASWFRGLGNL RVLDLSENFL
YKCITKTKAF QGLTQLRKLN LSFNYQKRVS FAHLSLAPSF
GSLVALKELD MHGIFFRSLD ETTLRPLARL PMLQTLRLQM
NFINQAQLGI FRAFPGLRYV DLSDNRISGA SELTATMGEA
DGGEKVWLQP GDLAPAPVDT PSSEDFRPNC STLNFTLDLS
RNNLVTVQPE MFAQLSHLQC LRLSHNCISQ AVNGSQFLPL
TGLQVLDLSH NKLDLYHEHS FTELPRLEAL DLSYNSQPFG
MQGVGHNFSF VAHLRTLRHL SLAHNNIHSQ VSQQLCSTSL
RALDFSGNAL GHMWAEGDLY LHFFQGLSGL IWLDLSQNRL
HTLLPQTLRN LPKSLQVLRL RDNYLAFFKW WSLHFLPKLE
VLDLAGNQLK ALTNGSLPAG TRLRRLDVSC NSISFVAPGF
FSKAKELREL NLSANALKTV DHSWFGPLAS ALQILDVSAN
PLHCACGAAF MDFLLEVQAA VPGLPSRVKC GSPGQLQGLS
IFAQDLRLCL DEALSWDCFA LSLLAVALGL GVPMLHHLCG
WDLWYCFHLC LAWLPWRGRQ SGRDEDALPY DAFVVFDKTQ
SAVADWVYNE LRGQLEECRG RWALRLCLEE RDWLPGKTLF
ENLWASVYGS RKTLFVLAHT DRVSGLLRAS FLLAQQRLLE
DRKDVVVLVI LSPDGRRSRY VRLRQRLCRQ SVLLWPHQPS
GQRSFWAQLG MALTRDNHHF YNRNFCQGPT AE

In one embodiment the anti-fibrotic agent can be TLR9 polypeptide or a functional fragment or variant of thereof, or a fusion thereof. Methods of synthesising TLR9 polypeptide would be routine to the person skilled in the art By “functional fragment or variant thereof” of SOCS 1, SOCS 3 and TLR9, we include a polypeptide that can be used as an anti-fibrotic agent in the invention. Such a variant may be encoded by a gene in which different codons can be substituted which code for the same amino acid(s) as the original codons. Alternatively, the substitute codons may code for a different amino acid that will not affect the function or immunogenicity of the protein or which may improve its function or immunogenicity. For example, site-directed mutagenesis or other techniques can be employed to create single or multiple mutations, such as replacements, insertions, deletions, and transpositions, as would be appreciated by the skilled person.

By “fusion thereof” we include where SOCS1, SOCS 3 or TLR9 polypeptide is fused to any other polypeptide or lipids. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well known Myc tag epitope.

Anti-fibrotic agents also include agents that increase the amount or activity of SOCS1, SOCS 3 or TLR9. Examples of such agents include agents that increase gene expression levels or translational efficiency of nucleic acid encoding SOCS1, SOCS 3 or TLR9.

By “increase the amount or function” we include where the agent increases the amount or function of SOCS 1, SOCS 3 or TLR9 by 2×, 3×, 5×, 10×, 20×, 50×, or 100× the natural amount or function.

In relation to this embodiment of the invention, the inventors have determined that SOCS 3 expression is decreased in fibrotic tissues due to methylation of the SOCS 3 gene. Therefore, agents that decrease methylation of the SOCS 3 locus, such as chromatin remodelling factors, can lead to an increase in SOCS 3 protein levels. Such agents could include modulators of DNA methylation such as azacitidine or zebularine.

Example 1 of the specification provides an assay by which the effect of a compound on fibrosis can be assessed. Therefore, the assay can be used as a screen to identify whether a test compound can be used as an anti-fibrotic agent in the invention. The assay can also be used to identify whether antibodies to the RhoA, RhoA GTPases, TGF-β1 and CTGF signalling molecules, or other members of the RhoA signalling pathway, act as antagonists, and whether mutated versions of these polypeptide can function as dominant inactive form of the molecule. Hence using this assay it would be possible for the skilled person to routinely identify further anti-fibrotic agents of use in the present invention.

A further embodiment of the invention is where the medicament further comprises, or the subject is administered an immunosuppressive and/or anti-inflammatory agent and/or a modulator of DNA methylation.

Examples of immunosuppressive and/or anti-inflammatory agents that can be used in this aspect of the invention include corticosteroids, azathioprine, cyclosprin, cyclophosphamide, methotrxate or hydroxychloroquine. Examples of modulators of DNA methylation include azacitidine, zebularine.

Medicaments in accordance with the invention may be formulated according to protocols well known in the art. Suitable formulations may be determined based on the preferred route by which the medicament is to be administered. Preferably medicaments according to the invention may be prepared in forms suitable for administration by inhalation, topical administration, ophthalmic administration, by injection, or by implantation.

As discussed above, preferably the fibrotic disorder to be treated by the use of the medicament or method of the invention is idiopathic pulmonary fibrosis. Also, preferably the anti-fibrotic agent is a statin compound or derivative thereof. In such an embodiment of the invention, preferably the statin or stain derivative is prepared as an aerosol for delivery intranasally or by inhalation to the lungs.

Statins or stain derivatives administered intranasally or by inhalation can be conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains a suitable quantity of statins or statin derivatives for delivery to the subject. It will be appreciated that the overall daily dose with an aerosol will vary from patient to patient, and may be administered in a single dose or, more usually, in divided doses throughout the day.

Where the statin or statin derivative is delivered to a subject as an aerosol, it is envisaged that the compound would be formulated as a nanoparticle. Nanoparticulated statins or statin derivatives may be prepared using techniques known in the art. For intranasal or inhalation delivery to the lung, it is preferred that the nanoparticulated statin or statin derivative has a diameter of less than 10 μm to achieve deposition in the distal airways and parenchyma. More preferably the nanoparticle has a diameter of 5 to 7 μm.

As discussed above, the fibrotic disorder to be treated by the use of the medicament or method of the invention may be fibrosis of the eye. Where the anti-fibrotic agent to be used is a statin compound or derivative thereof, in such an embodiment of the invention then the statin or statin derivative is preferably prepared for liquid delivery to the eye. For ophthalmic use, the statin or statin derivative can be formulated as micronised suspensions in isotonic, pH adjusted, sterile saline, or, preferably, as solutions in isotonic, pH adjusted, sterile saline, optionally in combination with a preservative such as a benzylalkonium chloride.

Furthermore, the fibrotic disorder to be treated by the use of the medicament or method of the invention may be fibrosis of the skin. Where the anti-fibrotic agent to be used is a statin compound or derivative thereof, in such an embodiment of the invention then the statin or statin derivative is preferably prepared for topical administration directly to the skin.

For application topically to the skin, the statin or statin derivative can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Where the medicament or method of the invention involves the use of biological cells, preferably the formulation for comprises biological cells in a suitable liquid carrier. Such a liquid carrier is preferably non-immunogenic, and may comprise a saline solution, cell culture medium, or distilled water. Formulations for injection may be as described above, or may also be provided in the form of a gel, which may preferably be capable of resolution by the body of the subject treated. Formulations suitable for implantation may take the forms described for injection or inhalation, and may also comprise biological cells provided in a scaffold or matrix capable of providing a foundation for new tissue development.

Where the medicaments of the invention do not comprise biological cells then further forms can be used in which to administer therapeutic agents. Thus, for example, the medicament may be in the form of a powder, tablet, capsule, liquid, ointment, cream, gel, hydrogel, aerosol, spray, micelle, transdermal patch, liposome or any other suitable form that may be administered to a person or animal. It will be appreciated that the vehicle should be one which is well tolerated by the subject to whom it is given.

Liquid vehicles are used in preparing solutions, suspensions, emulsions, syrups, elixirs and pressurized compositions. The liquid vehicle can contain suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by for example, intramuscular, intrathecal, epidural, intraperitoneal or subcutaneous injection. Sterile solutions can also be administered intravenously. The compounds may be prepared as a sterile solid composition which may be dissolved or suspended at the time of administration using sterile water, saline, or other appropriate sterile injectable medium. Vehicles are intended to include necessary and inert binders, suspending agents, lubricants, flavorants, sweeteners, preservatives, dyes, and coatings.

It will be appreciated that the amount of stem or progenitor cells, or of the agent capable of increasing the amount of endogenous stem cells and/or progenitor cells, or of the anti-fibrotic agent, to be used in the invention and thus formulated into a medicament, is determined by its biological activity and bioavailability which, in turn, depends on the mode of administration and the physicochemical properties of the agents or cells employed. The frequency of administration will also be influenced by the abovementioned factors, and particularly the half-life of the cells or agents within the subject being treated.

Optimal dosages to be administered may be determined by those skilled in the art, and will vary with the particular cells or agents in use, the strength of the preparation, the mode of administration, and the advancement of the disease condition that is to be treated. Additional factors depending on the particular subject being treated will result in a need to adjust dosages, including subject age, weight, gender, diet, and time of administration.

Where the anti-fibrotic agent is a statin or derivative thereof, the dosage of the compound used in the use of the medicament or method of the invention is preferably less than the dosage of that compound used to control hyperlipdaemia.

Known procedures, such as those conventionally employed by the pharmaceutical industry (e.g. in vivo experimentation, clinical trials etc), may be used to establish specific formulations of compositions and precise therapeutic regimes (such as daily doses of the agents or cells and the frequency of administration).

Daily doses may be given as a single administration (e.g. a daily tablet for oral consumption or as a single daily injection). Alternatively the cells or agents used may require administration twice or more times during a day, dependent of pharmacological, toxicological or efficacy studies.

A further aspect of the invention provides a composition comprising an agent capable of increasing the number of stem cells and/or progenitor cells available to and/or engraftment at a site of fibrosis and an anti-fibrotic agent.

A further aspect of the invention provides a pharmaceutical composition comprising an agent capable of increasing the number of stem cells and/or progenitor cells available to and/or engraftment at a site of fibrosis and an anti-fibrotic and a pharmaceutically acceptable carrier.

By “an agent capable of increasing the number of stem cells and/or progenitor cells available to and/or engraftment at a site of fibrosis” and “an anti-fibrotic agent” we include all embodiments of these features of the invention described above in relation to earlier aspects of the invention. Particularly preferred is where the composition and pharmaceutical composition of the invention include a therapeutically effective quantity of stem and/or progenitor cells.

Also preferred is where the anti-fibrotic agent is a statin compound or derivative thereof. In this embodiment it is preferred that the pharmaceutical composition is prepared as an aerosol for delivery intranasally or by inhalation to the lungs.

Therefore the composition and pharmaceutical compositions of the invention can comprise therapeutically effective quantity of stem and/or progenitor cells and statin compound or derivative thereof.

This invention also provides a process for making a pharmaceutical composition comprising combining an agent capable of increasing the number of stem cells and/or progenitor cells available to and/or engraftment at a site of fibrosis and an anti-fibrotic agent and a pharmaceutically acceptable vehicle.

In the practice of this invention the “pharmaceutically acceptable vehicle” is any physiological vehicle known to those of ordinary skill in the art useful in formulating pharmaceutical compositions.

In one preferred embodiment, the pharmaceutical vehicle may include a statin compound or derivative thereof and be formulated as an aerosol for delivery intranasally or by inhalation to the lungs.

A further aspect of the invention provides a composition or pharmaceutical composition as defined above in relation to earlier aspects of the invention for use in medicine.

An embodiment of the present invention will now be described, by way of example, with reference to the accompanying drawings, in which;

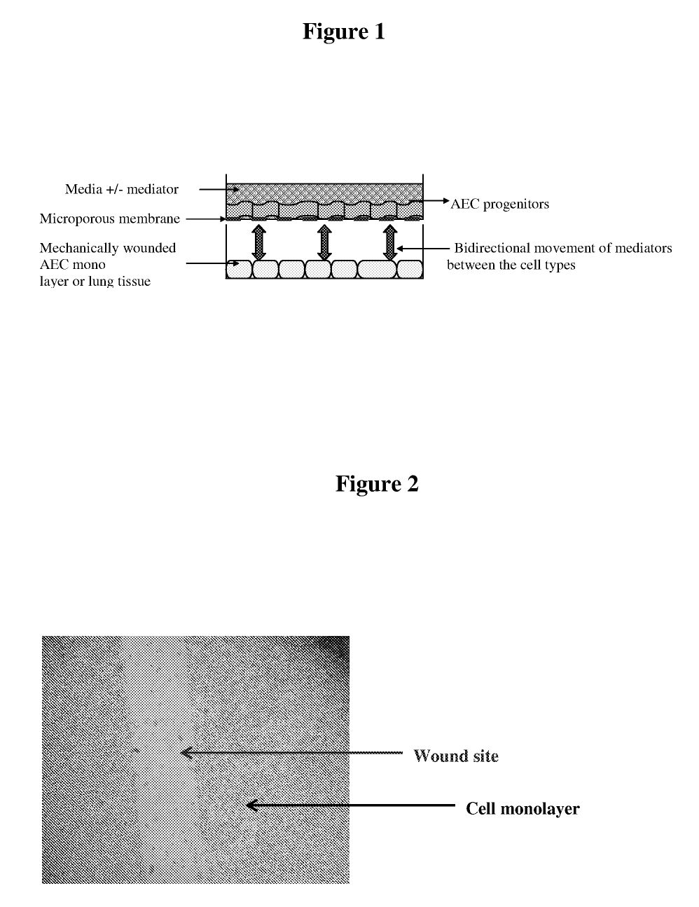

FIG. 1: Model of a screen to identify anti-fibrotic agents

FIG. 2: Image of alveolar epithelial cells wound repair model.

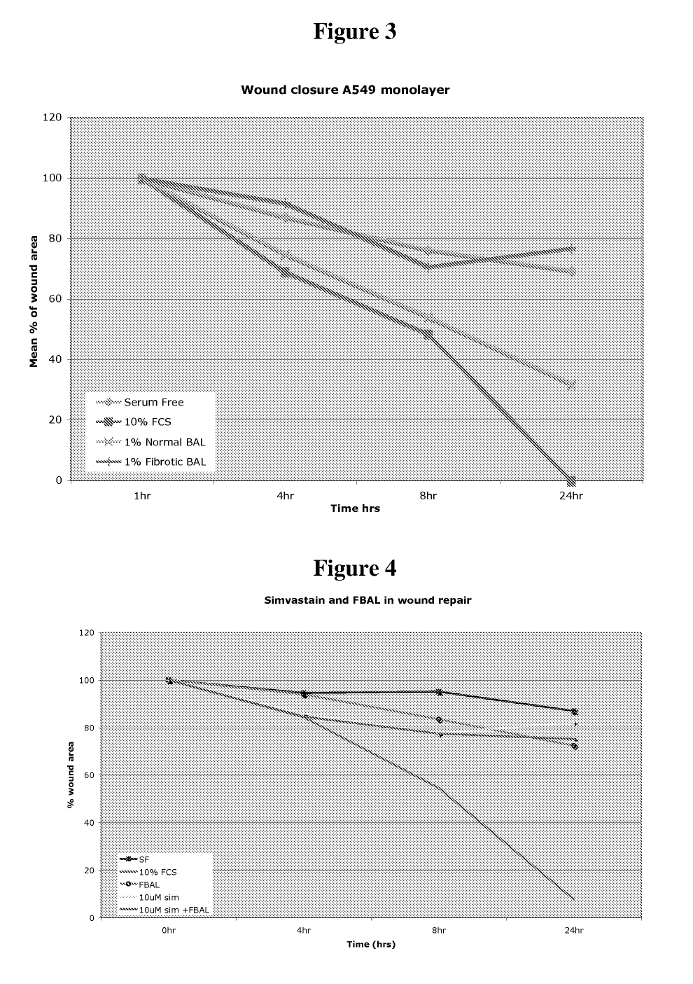

FIG. 3: Graph showing time to closure of epithelial cells wound.

FIG. 4: Effect of statin treatment on wound repair.

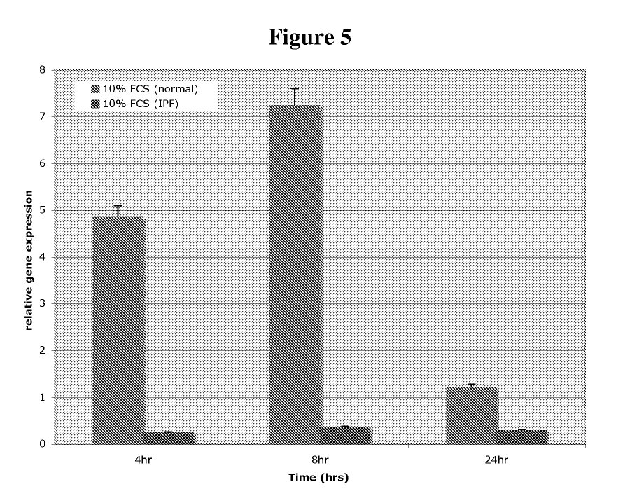

FIG. 5: SOCS3 gene expression in lung fibroblasts.

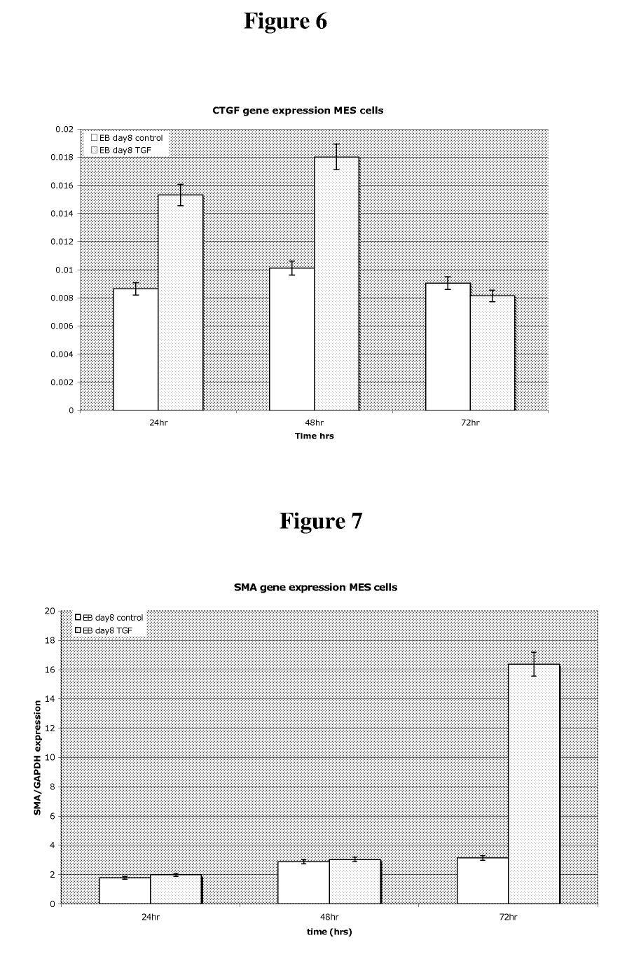

FIG. 6: CTGF gene expression during differentiation of murine ESCs.

FIG. 7: αSMA gene expression during differentiation of murine ESCs.

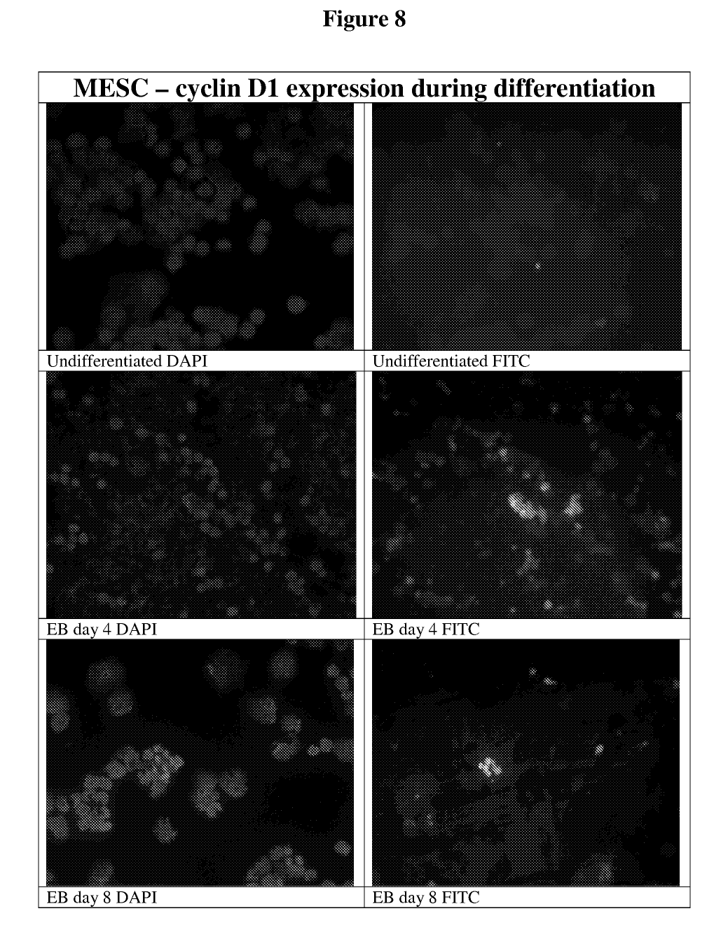

FIG. 8: Cyclin D1 and DAPI staining of differentiating stem cells.

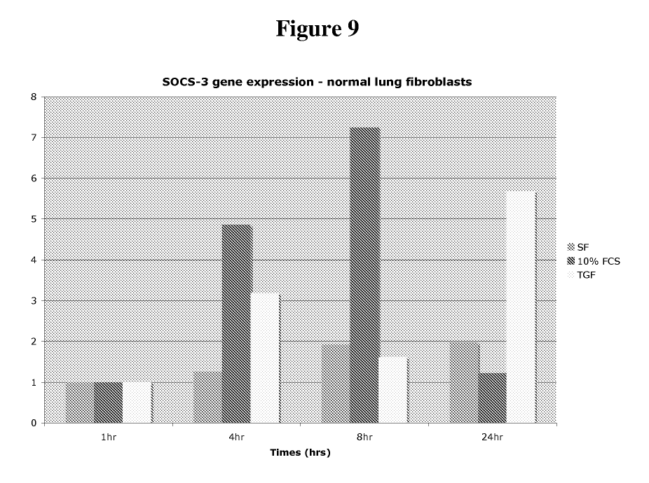

FIG. 9: SOCS-3 gene expression—normal lung fibroblasts.

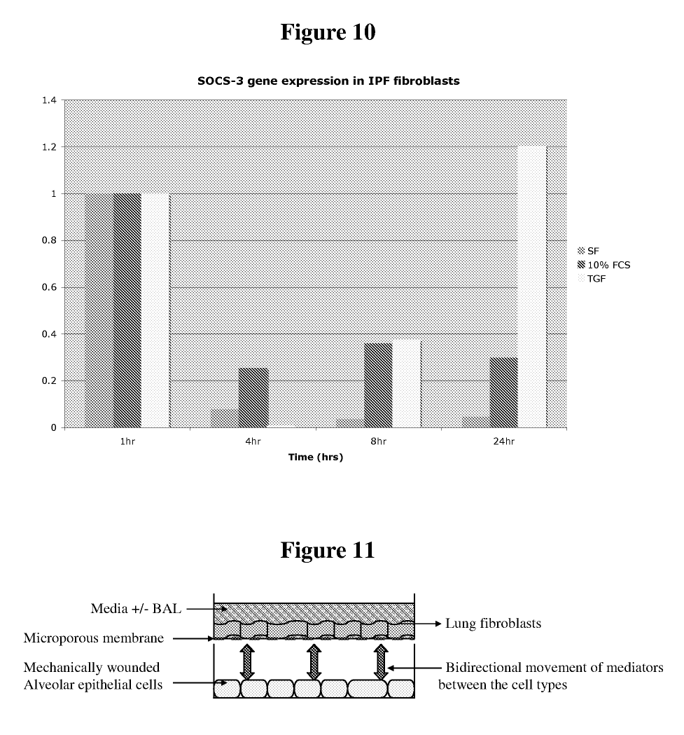

FIG. 10: SOCS-3 gene expression in IPF lung fibroblasts.

FIG. 11: Indirect contact model.

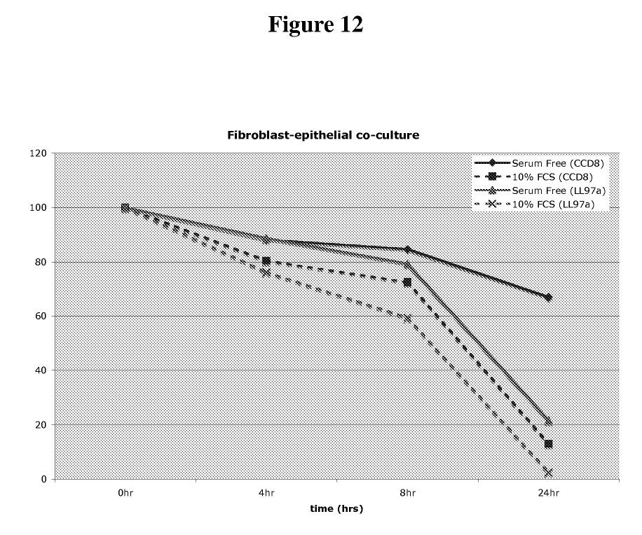

FIG. 12: Wound closure responses of A549 following co-culture with lung fibroblasts.

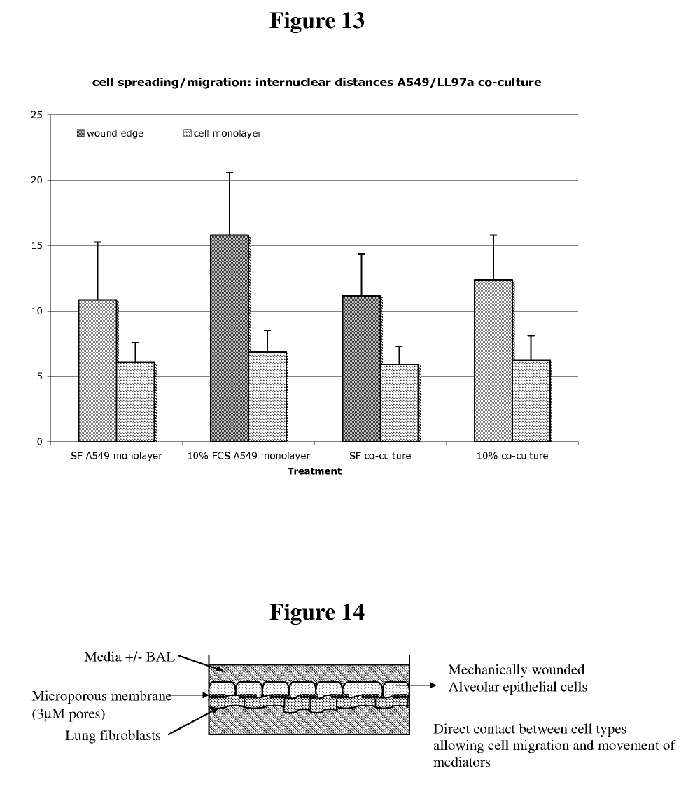

FIG. 13: Cell spreading/migration of A549 AEC during indirect co-culture with IPF derived lung fibroblasts.

FIG. 14: Direct contact model.

### EXAMPLE 1

### Assay for Evaluating Agents for Anti-Fibrotic Properties

Purpose of assay: to evaluate factors affecting ESC migration, engraftment and repair of a wounded alveolar epithelial layer.

The assay presented below can be used as a screen to identify anti-fibrotic agents as well as the amount of an agent that can be used to achieve an anti-fibrotic effect.

A key challenge for future successful stem cell-based strategies in pulmonary fibrosis is how interactions between progenitor cells, fibrotic stimuli and injured alveolar epithelium could influence effectiveness of wound repair. To address this, we use a specifically adapted alveolar epithelial wound model, to explore: (i) whether damaged epithelium is sufficient to direct stem cell differentiation; if not, whether ESC need to be differentiated and/or pre-purified from non-target cell populations; (ii) how such interactions could be modulated by superimposed presence of fibrotic stimuli. As a first step, this model offers a controlled, quantitative physiological analysis of ESC engraftment without the potential complications associated with an in vivo model e.g. clearance of implanted cells to extra-pulmonary tissues and immunogenicity. Information obtained will inform future in vivo ESC implantation protocols. ESC populations are seeded into transwell culture inserts, placed above monolayers of alveolar AEC (which can be wounded mechanically) (FIG. 1); the cell milieu can be selectively altered by conditioning with test stimuli, e.g. growth factors and/or bronchoalveolar lavage fluids from animal/patient models. Separate studies will incorporate thinly sliced harvested lung tissue from same mice instead of the AEC monolayer.

ESC populations studied can include (i) undifferentiated cells; (ii) differentiated according to AEC progenitor protocols but unpurified; (iii) differentiated, purified AEC progenitors. ESC are labelled with a generic fluorescent cell tracker dye, CDFA-SE, for time series visualization in real time using laser scanning confocal microscopy to track their migration and engraftment into epithelial cell monolayer or lung tissue. Analysis includes (a) quantification of engraftment and migration: epithelial cells/tissue containing engrafted ESC fixed in formalin; tissue is cryosectioned. Cell nuclei are visualized with DAPI; fluorescence microscopy is used to quantify number of migrated cells; (b) Phenotyping: migrated/engrafted cells are phenotyped by immunofluorescence for markers of differentiated lung epithelium (SPC, SPB, SPA, CC10, aquaporin 5); mesenchyme (α-SMA, pro-collagen I and III, vimentin, prolyl-hydroxylase-β); endothelium (CD31); macrophages (CD68); and undifferentiated ESC (October4, CD9). Signs of epithelial wound closure and rate of repair are also evaluated under the set conditions. Detection of apoptotic cells in wounded cell/tissue layers is analysed using annexin V-cy3. Fluorescent and phase images are captured and fluorescent apoptotic cells expressed as a percentage of the total number of cells present.

An agent to be tested for anti-fibrotic activity (indicated in FIG. 1 as a “mediator”) is supplied to the media. The effect of that agent on fibrosis can then be assessed by comparing the degree of fibrosis shown in the assay with the test agent to one or more “control” assays in which the test agent is omitted from the media. As would be appreciated by the skilled person, such an assay may also be of use in determining the amount of an agent that can be used to provide an “anti-fibrotic” effect.

Thus, the procedure described above can be utilised as an assay by which the effect of a compound on fibrosis can be assessed. Therefore, the assay can be used as a screen to identify whether a test compound can be used as an anti-fibrotic agent in the invention. The assay can also be used to identify whether antibodies to the RhoA, RhoA GTPases, TGF-β1 and CTGF signalling molecules, or other members of the RhoA signalling pathway, act as antagonists, and whether mutated versions of these polypeptide can function as dominant inactive form of the molecule. Hence using this assay it would be possible for the skilled person to routinely identify further anti-fibrotic agents of use in the present invention.

### EXAMPLE 2

### Data from an Alveolar Epithelial Cell Wound Repair Model

1. Data Demonstrating that Bronchoalveolar Lavage Fluid (BALF) from Patients with Lung Fibrosis Inhibits Epithelial Repair.

In the present studies an alveolar epithelial cells wound repair model was employed. Here, a monolayer of A549 alveolar epithelial cells (AEC) was mechanically wounded (scratched with a pipette tip; FIG. 2 shows the wound site in the epithelial monolayer) and incubated with BALF isolated from healthy, non-smoking volunteers or patients suffering from idiopathic pulmonary fibrosis (IPF). Closure of this wound was monitored over 24 hours following treatment.

Using this model, we demonstrated (FIG. 3) that factors present in fibrotic BAL significantly inhibits repair (p<0.05), compared to lavage from normal controls. Mechanisms for the inhibition of epithelial repair have also been investigated. We have demonstrated that IPF BAL inhibits cell spreading and migration by 2.6 fold. In addition, factors from a fibrotic BAL significantly abrogates the number of proliferating cells at the wound edge (p<0.05).

From this data we propose that pro-fibrogenic growth factors, such as CTGF, TGF, ET-1, within the fibrotic lung as represented in our model by BAL successful inhibit repair and/or regeneration of the lung alveolar epithelium. This inhibition of repair or regeneration perpetuates lung fibrosis. Our data demonstrates that it is necessary to modulate the fibrotic environment in the lung so that repair can be initiated.

2. Evidence that Statin Treatment is Able to Improve Epithelial Repair and Overcome the Inhibitory Effects Offibrotic BAL.

Using the AEC wound repair model described above, we examined whether statin-based compounds are able to improve alveolar epithelial repair. The data presented in FIG. 4 was derived using A549 epithelial cells and simvastatin. From this data it can be seen that statin improves wound closure in this model of wound repair.

### 3. Suppressor of Cytokine Signalling 3 (SOCS3) Expression is Abrogated in IPF Fibroblasts and May be a Novel Target for Treatment.

SOCS3 gene expression was measured in lung fibroblasts subjected to 10% FCS or TGFβ (5 ng/ml) by real time PCR. The results (FIG. 5) show that SOCS3 expression is virtually lost in IPF-derived lung fibroblasts, suggesting that these cells are hyper-responsive to overexpression of growth factors such as TGFβ.

We have determined that SOCS3 expression is reduced in IPF-derived lung fibroblast due to SOCS3 gene silencing by DNA methylation. From this finding we can suggest that increasing SOCS3 levels may function to reduce the lung fibrosis. Mechanisms of increasing SOCS3 levels include: introducing SOCS3 recombinant protein to kung epithelial cells; increasing SOCS3 gene expression by reducing methylation of SOCS 3; and increasing the amount of SOCS3 receptor protein.

Further information concerning SOCS3 and lung fibrosis is presented below.

### EXAMPLE 3

### Stem Cells and Fibrosis

1. Stem Cells Express Key Fibrogenic Mediators in Response to TGFβ—Suggesting that Stem Cells May Take Part and Perpetuate Fibrosis in a Fibrogenic Milieu.

We measured gene expression levels of key fibrogenic markers in stem cells when exposed to TGFβ. The results demonstrate that, during differentiation and development, stem cells express Connective Tissue Growth Factor (CTGF) (FIG. 6), and α-smooth muscle actin (SMA) (FIG. 7), both of which are key markers for the differentiation of stem cells into fibrotic and myofibroblast cell phenotypes.

### 2. Stem Cell Proliferation and Cell Cycle Regulation is Modulated by Cyclin D1 Depending on the Differentiation State.

Expression of cyclin D1, a recognised cell cycle regulator, has been implicated in fibrosis. Accordingly, using immunohistochemical staining we examined cyclin D1 levels during stem cell differentiation. As can be seem in FIG. 8, cyclin D1 expression levels varies during cell differentiation: it is expressed in stem cells day 4 and 8 but is not involved in stem cell proliferation in the undifferentiated phase. DAPI staining is used as a control to stain the nucleus of all the cells present in the field of view.

The data presented in FIGS. 6 to 8 demonstrates that, where stem cells are present in an environment that is bias towards fibrosis, which is the situation in the lungs of subjects suffering from IPF, then stem cells can propagate along fibroblast/myofibroblast lineage, away from the alveolar epithelial cell differentiation that is essential for lung repair.

### 4. Discussion

The findings set out above support a combinatorial approach to treat pulmonary fibrosis. Firstly, using statins or statin derivatives we can aborting, reversing or slow the pro-fibrotic milieu within the lungs (e.g. by reducing growth factor expression and/or release), and thus downregulate fibroblast proliferation, collagen synthesis and extracellular matrix deposition. Similar results can be achieved by manipulation of specific antifibrotic molecular targets, e.g SOCS3. Then stem cell therapy could be used to encourage regenerative capability of the damaged alveolar epithelium.

### EXAMPLE 4

### SOCS-3 as a Novel Mediator of Lung Fibrosis

### BACKGROUND

IPF is a distinct chronic fibrosing lung disorder of unknown etiology; prognosis is invariably poor and mean survival post-diagnosis is 2.9 years. Response to conventional therapeutic agents is extremely unusual. Unique histological hallmarks of fibroblastic foci formation and alveolar epithelium disruption are driven by intricate cellular and molecular events, including growth factor overexpression and aggressive myofibroblast proliferation. We have demonstrated in recent studies the anti-fibrotic potential of Simvastatin (recognised for its antilipidemic actions) in over-riding Transforming Growth Factor (TGF)-β-Connective Tissue Growth Factor (CTGF) interactions via a Rho signalling pathway¹, thus abrogating cellular pro-fibrotic differentiation². We have now determined that SOCS 3 gene expression is significantly inhibited in IPF-derived lung fibroblasts (p<0.05) suggesting that these cells will be “hyper-responsive” to growth factors and cytokines known to be overexpressed in IPF. While not wishing to be bound by any theory, we propose that deregulated expression of SOCS-1 and SOCS-3 perpetuates fibrogenic disease; subsequent modulation of SOCS proteins and pathways may restore some essential negative feedback mechanisms helping control fibrosis.

### Methodology:

### Cell Culture of Human Lung Fibroblasts.

Lung fibroblast cell lines (CCD8LU—normal adult lung fibroblasts, and HIPF, LL29 and LL97a—IPF-derived lung fibroblasts) were cultured in Dulbecco's Modified Eagle's Medium (DMEM; Gibco-BRL, Paisley, UK). The medium was supplemented with 1% antibiotic/antimycotic solution (10,000 units penicillin, 10 mg streptomycin, 25 μl amphotericin B per ml; Gibco-BRL), L-glutamine (2 mM, Gibco-BRL) and 10% foetal calf serum (FCS, Labtech, Sussex, UK). All procedures with the cells were carried out under aseptic conditions using sterile products in a class II laminar flow cabinet (BSB 4a flow cabinet, Gelaire Flow Laboratories, Buckinghamshire, UK). The cells lines were cultured at 37° C., in 5% humidified CO₂ and in a Galaxy B incubator (Scientific Laboratory Supplies Ltd). The fibroblast cultures were serum deprived for 48 hours prior to exposure of human recombinant Transforming Growth Factor (TGF)-β at 5 ng/ml for up to 24 hours.

### Real Time RT-PCR of SOCS3.

SOCS 3 gene expression was assessed in each cell type and condition using 2 μl of cDNA sample. The primer and probe sets were “predesigned assay on demand” probes (Applied Biosystems, Foster City, Calif.); thus they are designed, tested and standardised by the manufacturer to allow reproducible expression analysis. The primer and cDNA were added to the TaqMan universal PCR master mix (Applied Biosystems), containing the necessary reagents for PCR, and the reaction volume was made up with nuclease free water (Applied Biosystems). The real time PCR was the performed on the ABI prism 7,000 system (Applied Biosystems). Each sample was tested in duplicate; the mean quantity of target gene expression was determined using the relative standard curve method of analysis. The expression of SOCS 3 was normalised against the expression of the housekeeper gene β-actin. This data is presented in FIGS. 5, 9 and 10.

### CONCLUSIONS

We have demonstrated a significantly reduced gene expression of SOCS 3 in IPF derived lung fibroblasts compared to normal lung fibroblasts (p<0.05) {FIG. 5]. Thus indicating that IPF derived lung fibroblasts are hypersensitive to growth factors and cytokines (such as TGF-β) that are known to be highly upregulated in the fibrotic lung. The expression of SOCS 3 is altered in response to growth factors and serum although the magnitude of induction is significantly higher (6 fold higher induction) in the normal lung fibroblasts compared to IPF-derived lung fibroblasts [FIG. 9 and FIG. 10]. Thus the SOCS-3 expression is significantly lower in fibroblasts derived from a fibrotic lung and these same fibroblasts are less responsive to stimulation from key fibrogenic growth factors such as TGF-β and serum. Thus deregulation of SOCS 3 and its pathways may have pathological implications perpetuating fibrotic lung disease.

### REFERENCES

1. Watts K L, Sampson E M, Schultz G S, Spiteri M A. Am J Respir Cell Mol Biol. 2005; 32(4): 290-300
2. Watts K L, Spiteri M A. Am J Physiol Lung Cell Mol Physiol. 2004; 287(6): L1323-32.
3. Wormald S, Zhang J G, Krebs D L et al. JBC. 2006. 281(16): 11135-11143
4. Ogata H, Chinen T, Yoshida T et al. Oncogene. 2006; 25(17): 2520-253

### EXAMPLE 5

### Further Data Concerning the Epithelial Wound Repair Model

### In Vitro Epithelial Wound-Healing Assay

The wound healing assays were performed utilising A549 alveolar epithelial cells. The epithelial repair activity was determined using an in-vitro epithelial wound-healing assay as described in Example 1 (Geiser T et al. Am J Respir Crit. Care Med. 2001; 163: 1384-1388) using human A549 alveolar epithelial-like cells (American Type Culture Collection, Rockville, Md.). Briefly, A549 epithelial cells were cultured to confluency in 12-well plates for 48 hours in minimal essential medium (MEM) containing 10% fetal bovine serum and then mechanically wounded with a pipette tip. Concentrated BALF from each patient was added to the wounded epithelial monolayers (experiments performed in triplicates) and the area of the wound was measured over a time period of 24 hours.

Preliminary experiments show that the optimal range of wound-healing activity (50% of maximal effect) was obtained at a 100-fold dilution of the BALF. The rate of alveolar epithelial repair was expressed as % reduction in wound area and results were compared with the rate of epithelial repair obtained with the same dilution (100-fold) of BALF from healthy donors (control). In addition the mechanisms of wound repair we analysed; cell spreading/migration and cell proliferation were determined as described previously Geiser T et al. Am J Physiol: 2000; 279: 1184-1190.

### Co-Culture Studies:

Co-culture models involving A549 AEC and lung fibroblasts (normal adult lung fibroblasts CCD8 and IPF derived lung fibroblasts LL29, LL97a, HIPF) were designed as outlined below.

a) Indirect contact model (FIG. 11): Lung fibroblasts (normal or IPF derived) were seeded into a transmembrane well (0.8 μM pore size) to allow the movement of soluble factors between the cell types. A549 AEC seeded into a 12 well plate and mechanically wounded as described above. The wound closure of the AEC cells monitored over 24 hours.

The results shown in FIG. 12 suggest that co-culturing of AEC with fibroblasts in a non-direct model influences epithelial wound repair. As expected co-culturing in 10% FCS produced near complete closure of the wound site. However in serum free conditions co-culturing affects repair; IPF-derived lung fibroblasts producing a significantly (p>0.05) enhanced repair response at 24 hours compared to normal control lung fibroblasts (CCD8).

Mechanisms of repair were investigated by measuring the internuclear distances of the cells at the wound edge and within the cell monolayer. The internuclear distances of the cells remain unchanged within the cell monolayer and at the wound edge regardless of co-culture conditions (see FIG. 13).

b) Direct contact model (FIG. 14). A549 AEC were seeded inside the transwell (pore size 3 μM, 100,000 cell per insert) and grown until confluent. Then turn the transwell upside down and rest in a Petri dish and carefully seed the fibroblast (100,000 cell per insert) onto the lower surface of the membrane and leave in the incubator for 4 hours to allow adherence. The cell attachment was verified and the excess media carefully removed. The transwell on which the fibroblasts and epithelial cells had been seeded was placed into a clean 12 well plate and top and bottom sections filled with media (MEM) allowing direct contact of the cells enabling cell migration and movement of soluble factors between the cell types. A mechanical wound (caused by scratching with a pipette tip) was carefully administered to the A549 AEC layer and wound closure was monitored over a 24 hours period. The soluble factors produced by the cell types are able to cross the membrane and the pores are also big enough to allow migration of the cells through the pores to interact with the wound repair processes.

The results of the direct contact model are presently being investigated. Optimisation of imaging techniques was performed which would allow dual staining of the cell types for imaging by confocal microscopy. All the cells will be labelled with rhodamine labelled phalloidin, so to achieve successful imaging we required a fibroblast marker that would stain the fibroblast without non-specifically labelling the epithelial cells. This would allow us the assess whether there is any fibroblast migration across the membrane to participate in the repair process, and also image the wound closure of the alveolar epithelial cells at a fixed time point of 24 hours. Initial attempts to use vinculin-cy3 staining were unsuccessful as this antibody could not specifically stain the fibroblast population and caused significant non-specific staining in the epithelial cell type. The staining was optimised and successful labelling of the lung fibroblasts cells was achieved using a primary monoclonal antibody against prolyl hydroxylase beta (Novus) and a rhodamine labelled goat anti-mouse secondary antibody. Successful labelling of the lung fibroblasts (with no non-specific epithelial contamination) was successfully achieved using primary antibody concentrations ranging from 1 μg/ml-10 μg/ml followed by secondary antibody at 10 μl/ml.

What is claimed is:

1. A method comprising using an agent capable of increasing the number of stem cells and/or progenitor cells available to and/or engraftment at a site of fibrosis in the manufacture of a medicament for use in conjunction with an anti-fibrotic agent for the treatment of fibrosis.

2. A method of treating fibrosis comprising administering an agent capable of increasing the number of stem cells and/or progenitor cells available to and/or engraftment at a site of fibrosis in conjunction with an anti-fibrotic agent, to a subject in need of said administration.

3. The method of claim 2 wherein the agent capable of increasing the number of stem cells and/or progenitor cells available to and/or engraftment at a site of fibrosis is a therapeutically effective quantity of stem cells and/or progenitor cells.

4. The method of claim 3 wherein the therapeutic stem cells and/or progenitor cells are adapted to differentiate into alveolar epithelial cells.

5. The method of claim 2 wherein the agent capable of increasing the number of stem cells and/or progenitor cells available to and/or engraftment at a site of fibrosis increases the amount of endogenous stem cells and/or progenitor cells.

6. The method of claim 2 wherein the anti-fibrotic agent is supplied to the subject before, at the same time, or after the agent capable of increasing the number of stem cells and/or progenitor cells available to and/or engraftment at a site of fibrosis.

7. The method of claim 2 wherein the fibrosis is idiopathic pulmonary fibrosis, fibrosis of the eye or fibrosis of the skin.

8. The method of claim 7 wherein the fibrosis is idiopathic pulmonary fibrosis.

9. The method of claim 2 wherein the anti-fibrotic agent is a modulator of RhoA, RhoA GTPases, TGF-β1 or CTGF, or any other member of the RhoA signalling pathway, or modulates the effect of suppressor of cytokine signalling 1 (SOCS 1), suppressor of cytokine signalling 3 (SOCS 3) or TLR9.

10. The method of claim 2 wherein the anti-fibrotic agent is a statin compound or derivative thereof.

11. The method of claim 10 wherein the statin is lovastatin, pravastatin, fluvastatin, cerivastatin, atorvastatin, simvastatin, pitavastatin and rosuvastatin.

12. The method of claim 2 wherein the subject is also supplied with one or more of an immunosuppressive agent, an anti-inflammatory agent, a modulator of DNA methylation.

13. The method of claim 12 wherein the subject is supplied with two or more of an immunosuppressive agent, an anti-inflammatory agent, a modulator of DNA methylation.

14. A composition comprising an agent capable of increasing the number of stem cells and/or progenitor cells available to and/or engraftment at a site of fibrosis and an anti-fibrotic agent.

15. The composition of claim 14 further comprising a pharmaceutically acceptable carrier, wherein the composition is a pharmaceutical composition.

16. A composition as defined in claim 15 formulated as an aerosol for delivery intranasally or by inhalation to the lungs.

17. A process for making a pharmaceutical composition comprising combining an agent capable of increasing the number of stem cells and/or progenitor cells available to and/or engraftment at a site of fibrosis and an anti-fibrotic agent and a pharmaceutically acceptable vehicle.

18. A method comprising treating fibrosis by administering an anti-fibrotic agent in conjunction with a second therapeutic agent, the second therapeutic agent adapted to increase the quantity or engraftment of therapeutic cells at the site of fibrosis, the therapeutic cells selected from stem cells and progenitor cells.

19. The method of claim 18 wherein the anti-fibrotic agent is a statin compound or derivative thereof.

20. The method of claim 19 wherein the second therapeutic agent includes a therapeutically effective quantity of the therapeutic cells.

21. The method of claim 20 wherein the fibrosis is idiopathic pulmonary fibrosis, fibrosis of the eye or fibrosis of the skin.

22. The method of claim 18 wherein at least the second therapeutic agent is administered intranasally or by inhalation to the lungs.

23. The method of claim 22 wherein the anti-fibrotic agent is a modulator of RhoA, RhoA GTPases, TGF-β1 or CTGF, or any other member of the RhoA signalling pathway, or modulates the effect of suppressor of cytokine signalling 1 (SOCS 1), suppressor of cytokine signalling 3 (SOCS 3) or TLR9.


SEQUENCE LISTING (3 sequences)

SEQ ID NO: 1 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 2 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 3 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE